(19) United States
(12) Patent Application Publication
(10) Pub. No.: US 2026/0182964 A1
(43) Pub. Date: Jul. 2, 2026

(54) ULTRASOUND DIAGNOSTIC SYSTEM

(71) Applicant: FUJIFILM Corporation, Tokyo (JP)

(72) Inventor: Natsumi DANNO, Tokyo (JP)

(73) Assignee: FUJIFILM Corporation, Tokyo (JP)

(21) Appl. No.: 19/430,269

(22) Filed: Dec. 23, 2025

(30) Foreign Application Priority Data
Dec. 27, 2024 (JP) 2024-232134

(51) Int. Cl. A61B 8/00; A61B 8/06; G06T 7/00

(52) U.S. Cl. CPC A61B 8/463; A61B 8/06; A61B 8/488; A61B 8/54; G06T 7/0014; G06T 2207/10132; G06T 2207/30096; G06T 2207/30104

(57) ABSTRACT

A system generates an ultrasound image from echo signals obtained by a probe, analyzes the ultrasound image, generates association information including a result of the analysis or the like, and registers the ultrasound image and the association information in a database. The system searches the database for a prior ultrasound image having association information close to the association information. Further, the system calculates image similarity between a current ultrasound image and each of the searched prior images, calculates a relevance score from the image similarity and similarity of the association information, and extracts several prior images in descending order of the relevance score. The extracted images are displayed in a list in a thumbnail display field.

### CROSS REFERENCE TO RELATED APPLICATION

This application claims priority to Japanese Patent Application No. 2024-232134 filed on December 27, 2024 which is incorporated herein by reference in its entirety including the specification, claims, drawings, and abstract.

### BACKGROUND

1. Technical Field

The present disclosure relates to an ultrasound diagnostic system.

### 2. Description of the Related Art

In ultrasound diagnosis, an ultrasound image obtained in a previous examination for the same subject as a current examination is displayed for reference.

An ultrasound diagnostic apparatus disclosed in JP2022-127808A stores an ultrasound image acquired during execution of steps in a protocol as a reference image. The ultrasound diagnostic apparatus displays a prior reference image stored in the same step for the same patient side by side with the ultrasound image acquired and displayed in the step that is currently being executed (for example, see paragraphs 0037 and 0055 to 0056 of JP2022-127808A).

An ultrasound diagnostic system disclosed in JP6663027B provides a first display screen on which thumbnails of a plurality of ultrasound images of a previously diagnosed subject are displayed in a list (see paragraphs 0046 and 0050 to 0053 of JP6663027B). Further, the ultrasound diagnostic system provides a second display screen on which one of a plurality of prior ultrasound images and the ultrasound image acquired in the current examination are displayed side by side (see paragraphs 0046 and 0054 to 0055 of JP6663027B). A user performs ultrasound diagnosis of a part corresponding to the prior image while viewing the prior image on the second display screen. The image acquired in the ultrasound diagnosis is displayed side by side with the prior image.

A medical image examination apparatus disclosed in JP3791849B searches for a prior image of the same patient captured under the same imaging conditions as imaging conditions of a current examination, and displays the prior image side by side with an image obtained in the current examination (see paragraphs 0026 and 0029 to 0033 of JP3791849B).

### SUMMARY

An aspect of the present disclosure relates to an ultrasound diagnostic system comprising: a processor configured to: generate a first ultrasound image from reception signals of an ultrasound probe; execute processing of displaying the first ultrasound image; execute an analysis of the first ultrasound image to obtain an analysis result; execute a search for prior ultrasound images having analysis results that are the same as or similar to the analysis result for the first ultrasound image; calculate a score representing similarity to the first ultrasound image for each of the prior ultrasound images obtained as a result of the search; and execute display control of displaying, together with the first ultrasound image, one or more images selected based on the scores from among the prior ultrasound images obtained as a result of the search.

The analysis may be, for example, processing of obtaining, as the analysis result, a cross-section recognition result indicating which of a plurality of predetermined cross sections the first ultrasound image corresponds to.

In one aspect, the first ultrasound image is an image of a blood-flow velocity waveform obtained by a Doppler method, and the analysis is processing of obtaining, as the analysis result, a maximum flow velocity of the blood-flow velocity waveform.

In another aspect, the processor is configured to: execute processing of detecting a tumor region from the first ultrasound image; and calculate, in the calculation of the scores, the score between the prior ultrasound image and the first ultrasound image based on first similarity between a whole prior ultrasound image and a whole first ultrasound image and second similarity between the tumor regions.

In still another aspect, the processor is configured to: in a case of storing the first ultrasound image, store the analysis result obtained by the analysis as one item of attribute information of the first ultrasound image.

In still another aspect, the processor is configured to: in a case in which the first ultrasound image is a moving image, store the moving image, a still image that is representative of the moving image, and an analysis result obtained for the still image by the analysis.

In still another aspect, the processor is configured to: in the search, acquire memo information input by a user for the displayed first ultrasound image, and search for prior ultrasound image data having an analysis result that is the same as or similar to the analysis result and having memo information that is the same as or similar to the acquired memo information.

In still another aspect, the processor is configured to: in the display control, execute processing of displaying thumbnails of a plurality of images selected based on the scores from among the prior ultrasound images obtained as a result of the search side by side in descending order of the score in a thumbnail display field provided near the first ultrasound image.

In still another aspect, the processor is configured to: in the display control, in a case in which an image designated as a key image is included in the plurality of images selected based on the scores from among the prior ultrasound images obtained as a result of the search, display a thumbnail of the image designated as the key image at a first position of the thumbnail display field regardless of the score for the image.

In still another aspect, the processor is configured to: in a case in which a protocol assistant function of supporting execution of a protocol consisting of one or more examination steps is used, in the display control, display a thumbnail of a prior ultrasound image obtained in the same examination step of the same protocol as an examination step of a protocol that is currently being executed, at the first position of the thumbnail display field, and display the thumbnail of the image designated as the key image at a next position.

The processor is configured to: in a case in which a protocol assistant function of supporting execution of a protocol consisting of one or more examination steps is used, in the display control, display a thumbnail of a prior ultrasound image obtained in the same examination step of the same protocol as an examination step of a protocol that is currently being executed, at a first position of the thumbnail display field.

In still another aspect, the processor is configured to: in the display control, display the prior ultrasound image having a highest score side by side with the first ultrasound image, as a reference image for the first ultrasound image.

In still another aspect, the processor is configured to: in a case in which information indicating a measurement performed on the prior ultrasound image displayed as the reference image is obtained, execute processing of performing the measurement on the displayed first ultrasound image.

In still another aspect, the processor is configured to: select a layout for displaying the first ultrasound image and a reference image selected from among the prior ultrasound images, based on the cross-section recognition result.

In still another aspect, the processor is configured to: in a case in which the cross-section recognition result corresponds to a predetermined first type of cross section, select a dual layout in which the first ultrasound image and the reference image are arranged in the same size.

In still another aspect, the processor is configured to: in a case in which a finding is generated by the analysis, as a layout for displaying the first ultrasound image and a reference image selected from among the prior ultrasound images, select a dual layout in which the first ultrasound image and the reference image are arranged in the same size.

In still another aspect, the processor is configured to: in a case in which a mode of the ultrasound diagnostic system when the first ultrasound image is generated is a specific mode, as a layout for displaying the first ultrasound image and a reference image selected from among the prior ultrasound images, select a dual layout in which the first ultrasound image and the reference image are arranged in the same size.

### BRIEF DESCRIPTION OF THE DRAWINGS

FIG. 1 is a diagram illustrating an example of a functional configuration of an ultrasound diagnostic system according to an embodiment.

FIG. 2 is a diagram schematically illustrating an example of a screen configuration of a display screen provided by the ultrasound diagnostic system.

FIG. 3 is a diagram schematically illustrating another example of the screen configuration of the display screen provided by the ultrasound diagnostic system.

FIG. 4 is a diagram schematically illustrating still another example of the screen configuration of the display screen provided by the ultrasound diagnostic system.

FIG. 5 is a diagram illustrating an item group included in the association information.

FIG. 6 is a diagram illustrating an item group belonging to imaging conditions in the association information.

FIG. 7 is a diagram illustrating an item group belonging to an analysis result in the association information.

FIG. 8 is a diagram illustrating an item group belonging to user input data in the association information.

FIG. 9 is a diagram illustrating a procedure of processing of searching for a prior image that is highly related to a live image.

FIG. 10 is a diagram schematically illustrating an image of a prior image region generated by the procedure of FIG. 9.

FIG. 11 is a diagram illustrating a changed portion of the procedure of FIG. 9 in a case of taking a key image into account.

FIG. 12 is a diagram schematically illustrating an image of a prior image region generated by the procedure of FIG. 11.

FIG. 13 is a diagram illustrating a changed portion of the procedure of FIG. 9 in a case of taking a protocol assistant function into account.

FIG. 14 is a diagram illustrating a changed portion of the procedure of FIG. 9 in a case of taking the key image and the protocol assistant function into account.

FIG. 15 is a diagram schematically illustrating an image of a prior image region generated by the procedure of FIG. 14.

FIG. 16 is a diagram illustrating a changed portion of the procedure of FIG. 9 in a case of taking similarity between tumor regions into account.

FIG. 17 is a diagram illustrating a processing procedure in a case of registering a moving image in a database.

FIG. 18 is a diagram illustrating a processing procedure of controlling a measurement in conjunction with display of a reference image.

FIG. 19 is a diagram illustrating an example of a procedure of automatically selecting a layout of the display screen.

### DETAILED DESCRIPTION

Hereinafter, an embodiment of the present disclosure will be described with reference to the accompanying drawings.

### Example of Functional Configuration of Ultrasound Diagnostic System

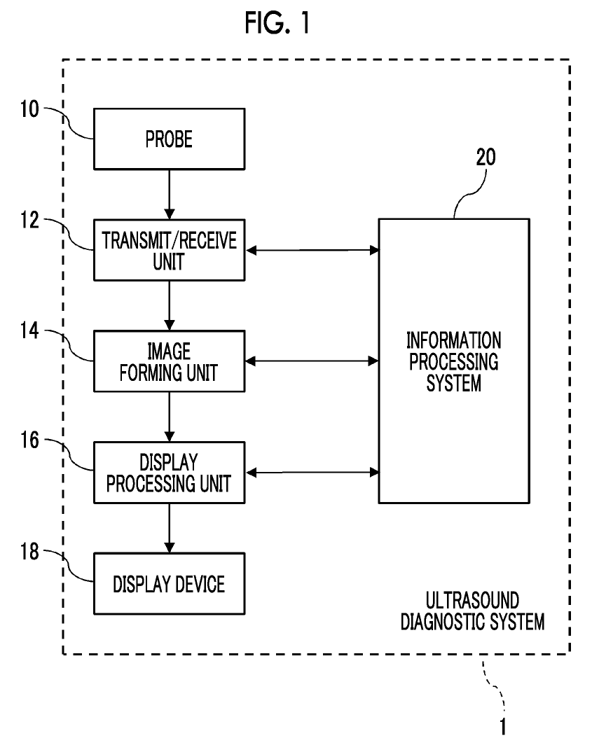

FIG. 1 illustrates an ultrasound diagnostic system 1 according to the embodiment. The ultrasound diagnostic system 1 is a medical system that is installed in a medical institution and is used for an ultrasound examination. The ultrasound diagnostic system 1 comprises a probe 10, a transmit/receive unit 12, an image forming unit 14, a display processing unit 16, a display device 18, and an information processing system 20 as functional elements.

The probe 10 transmits and receives ultrasound. The probe 10 includes a transducer element array consisting of a plurality of transducer elements. An ultrasound beam is formed by the transducer element array and is electronically scanned. As a result, a beam scanning surface is formed.

The transmit/receive unit 12 supplies a plurality of transmission signals to the transducer element array in parallel during transmission, and applies coherent summation (delay-and-sum) to a plurality of reception signals from the transducer element array during reception. As a result, beam data is formed. One reception frame data is generated by one scanning with the ultrasound beam. One reception frame data is formed by a plurality of beam data arranged in an electronic scanning direction. Each beam data is formed by a plurality of echo data arranged in a depth direction.

The reception frame data sequentially output from the transmit/receive unit 12 is sequentially input to the image forming unit 14. The image forming unit 14 includes a digital scan converter (DSC). The DSC generates a display frame data sequence from a reception frame data sequence. The DSC has a coordinate transformation function, a pixel interpolation function, and the like. One display frame data constitutes one tomographic image data. Although not illustrated in FIG. 1, the image forming unit that forms various ultrasound images is provided. The various ultrasound images include, for example, two-dimensional tomographic images such as a B-mode tomographic image, a color Doppler image (also referred to as a color flow image), and a power Doppler image. In addition, the ultrasound image to be formed may include an image formed by combining a plurality of types of two-dimensional tomographic images, such as an image in which a color Doppler image or a power Doppler image is superimposed on a B-mode tomographic image. In addition, an image (for example, a signal waveform or a temporal change in intensity) in a form other than the two-dimensional tomographic image, such as a pulsed-wave Doppler image, is also an example of the ultrasound image.

The display processing unit 16 has an image combining function, a color processing function, and the like. A display screen displayed on the display device 18 is formed by the display processing unit 16. First, the display screen includes the ultrasound image. The ultrasound image is a moving image during real-time operation and is a still image in a freeze state. The display device 18 is configured by an LCD, an organic EL display, and the like. The display screen includes various images generated by the information processing system 20. The various images may include a step list image, a measurement list image, a measurement value graph, an examination value graph, and the like.

The information processing system 20 executes information processing such as control of an operation of each component of the ultrasound diagnostic system 1, various types of analysis processing on the ultrasound image, generation of various images to be displayed on the display screen, and a layout configuration of the display screen. The information processing system 20 comprises, as hardware, for example, a processor such as a central processing unit (CPU) or a memory such as a random access memory (RAM).

### Example of Analysis Processing

Examples of the analysis processing executed by the information processing system 20 include the following.

### Cross-Section Recognition

The analysis processing executed by the information processing system 20 includes, for example, cross-section recognition. The cross-section recognition is processing of recognizing which cross section of a subject the ultrasound image (for example, the B-mode tomographic image) represents.

The cross section of the subject is specified by, for example, a combination of an organ such as a visceral organ in the subject and a positional relationship of the cross section with respect to the organ. In addition, in a guideline for ultrasound diagnosis for each part such as a heart, an abdomen, and a breast, a cross section to be imaged in the diagnosis of the part is determined.

For the cross-section recognition, for example, a machine learning model is used. The machine learning model may be constructed by, for example, a method such as a neural network. In order to construct the machine learning model, for example, a large number of pairs of the ultrasound image and information (for example, an identification name of the cross section) that specifies the cross section represented by the ultrasound image are prepared as training data. Then, the machine learning model is trained by providing the ultrasound image as input data and providing the information that specifies the cross section as labeled data to the machine learning model. By installing the machine learning model that has been sufficiently trained in this way in the information processing system 20, the information processing system 20 has a cross-section recognition function. In a case in which the ultrasound image is input, the trained machine learning model determines the cross section indicated by the image and outputs information for specifying the determined cross section. In addition, the machine learning model can output a confidence level of the determined cross section.

In addition, as in the cross-section recognition, a machine learning model capable of recognizing a visceral organ displayed in the ultrasound image can be constructed and used.

### Flow Velocity Calculation

The flow velocity calculation is processing of calculating a flow velocity of a blood flow flowing through a blood vessel. In this processing, the flow velocity of the blood flow is calculated by analyzing the ultrasound image including flow velocity information, such as a color Doppler image or a pulsed-wave Doppler mode (also referred to as a PW mode) image.

A target part for the flow velocity calculation may be designated by a user or may be automatically set by the information processing system 20. Examples of the designation by the user include a case in which the user designates a sample volume in the pulsed-wave Doppler mode on the B-mode tomographic image. In addition, in an example in which the information processing system 20 automatically sets the part, for example, a part in which the flow velocity is measured for each cross section is automatically set for each cross section. Then, the information processing system 20 specifies a measurement part set for the cross section represented by the ultrasound image that is currently being displayed, and calculates the flow velocity of the part from the ultrasound image or the like.

The flow velocity to be calculated may be a maximum flow velocity or an average flow velocity in the measurement target part. Which flow velocity to calculate is set in advance.

In one example, the information processing system 20 calculates a maximum flow velocity in a blood-flow velocity waveform obtained in the PW mode as an analysis result. In another example, the information processing system 20 calculates an average flow velocity in a region of interest (which may be designated by the user) in the color Doppler image as the analysis result.

### Tumor Detection

The tumor detection is processing of detecting a region having characteristics of a tumor from the ultrasound image such as the B-mode tomographic image.

For the tumor detection, for example, a machine learning model is used. The machine learning model may be constructed by, for example, a method such as a neural network. In order to construct the machine learning model, for example, a large number of pairs of the ultrasound image and information on the tumor for the ultrasound image are prepared as training data. Then, the machine learning model is trained by providing the ultrasound image as input data and providing the information on the tumor as labeled data to the machine learning model. By installing the machine learning model that has been sufficiently trained in this way in the information processing system 20, the information processing system 20 has a tumor detection function. The information on the tumor may be a response indicating whether or not the tumor is present. In this case, the trained machine learning model determines whether or not the region having the characteristics of the tumor is included in the input ultrasound image, and outputs a result of the determination together with the confidence level. In addition, the information on the tumor may be information representing a position and a range of the region having the characteristics of the tumor included in the ultrasound image. In this case, the trained machine learning model can determine the position and the range of the region having the characteristics of the tumor from the input ultrasound image, and output the result of the determination and the confidence level. Such a tumor detection function is practically used, for example, as computer-aided detection (CAD).

### Others

In addition, the information processing system 20 does not need to perform all types of the analysis processing described above, and may execute analysis processing other than the analysis processing described above. For example, as another analysis processing, the information processing system 20 may specify a part of the blood vessel to be measured in a specific cross section on the B-mode tomographic image, and automatically calculate a diameter of the blood vessel at the part. In addition, as another analysis processing, the information processing system 20 may extract an image feature value such as a histogram, an edge, or a texture from the B-mode image or the color Doppler image. These image feature values can also be used for calculating the image similarity described later for the B-mode image or the color Doppler image. In addition, as another analysis processing, the information processing system 20 may extract a feature value of the blood-flow velocity waveform from the pulsed-wave Doppler mode image. The feature value may be used for calculating the image similarity for the pulsed-wave Doppler mode image.

In addition, as another example of the analysis processing, there is processing of generating a finding. The finding is a sentence describing an abnormality or a symptom thereof detected by the examination. For example, in a case in which a tumor-suspected part is detected by a tumor detection function, a finding indicating that there is a suspicion of the tumor is generated. In a case in which the abnormality or the symptom thereof is not detected at all from the ultrasound image, the finding is not generated. In order to automate the generation of the finding, for example, a machine learning model that generates the finding from one or more information among the ultrasound image, results of various types of analyses on the ultrasound image, or a detection result of another detection system such as an electrocardiogram may be constructed and used. The finding generated by the information processing system 20 is checked by the user such as a doctor, is corrected as necessary, and then registered in the database.

### Protocol Assistant Function

The information processing system 20 may have a protocol assistant function. The protocol assistant function is a function of supporting the execution of a flow of a series of examinations that tend to be complicated, by automatically executing a protocol (that is, an examination procedure). The protocol defines a procedure of the series of examinations executed by using the functions of the ultrasound diagnostic system 1. For example, the protocol defines the content (for example, the cross section to be examined or imaging conditions) of each examination step to be executed and an execution order of the examination steps.

In a case in which a specific protocol is selected and executed, an execution condition set in advance for each step is automatically set. In addition, in a case in which the step is executed, necessary measurement is initiated. In a case in which a store operation is performed, the processing automatically transitions to the next step. Each step constituting the examination protocol is also referred to as a view from the perspective of image observation.

In the information processing system 20, a standard protocol suitable for each intended use is installed for each intended use. In addition, a dedicated protocol for a patient can be created by partially modifying the standard protocol, and then registered in the information processing system 20 and reused. For example, the mechanism disclosed in JP2022-127808A can be used to use the standard protocol or the dedicated protocol.

### Example of Display Screen

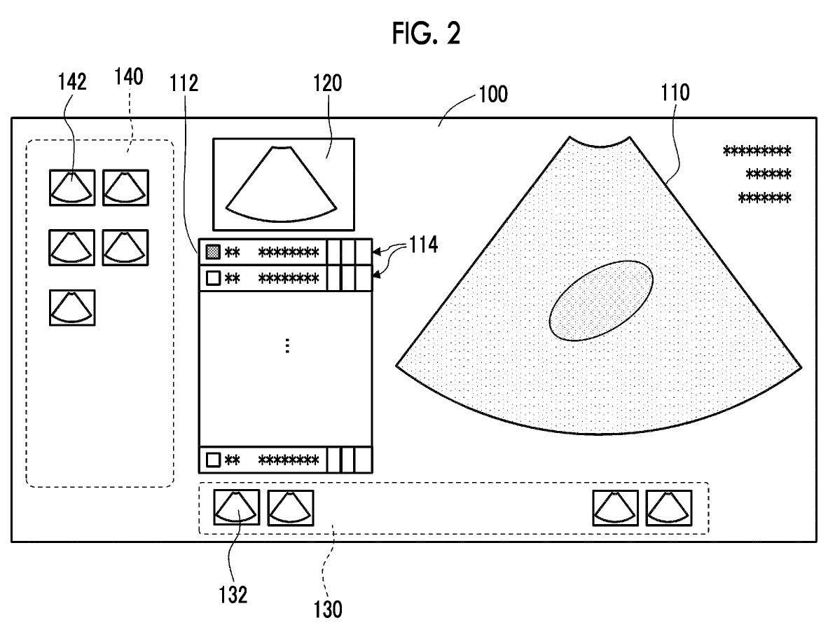

FIG. 2 illustrates an example of a display screen 100 configured in the information processing system 20.

A live image 110 is displayed on the display screen 100. The live image 110 is a live ultrasound image acquired by the ultrasound transmission and reception using the probe 10. In a case in which a specific protocol is selected, a step list 112 indicating the content of the specific protocol is displayed. The step list 112 is configured by a plurality of step displays 114 corresponding to a plurality of steps. The step display 114 corresponding to the step that is currently being executed is displayed in a form (for example, a different background color from other steps) that is distinguishable from the step displays corresponding to other steps. Although not illustrated, a measurement list may be displayed in which information (measurement name or the like) that specifies one or a plurality of measurements defined for the step that is currently being executed is displayed. A measurement value display field may be provided in which the acquired measurement value is displayed.

A reference image 120 is displayed near the live image 110. The reference image 120 is an image for reference for interpreting the live image 110. In a typical example, the reference image 120 is selected from among the prior images. The prior image is an ultrasound image previously acquired from the same subject as in the live image 110 and registered in the database. For example, the reference image 120 is a prior image of the same type as the live image 110 (for example, the ultrasound image of the same mode for the same cross section as the live image 110). However, this is merely an example.

In a case in which the prior image is displayed as the reference image 120, the information processing system 20 searches the database for the same type of image to be displayed as the reference image 120 based on the live image 110 and various attribute information (hereinafter, also referred to as association information) thereof.

For example, the information processing system 20 searches for the prior image associated with the same step identification information as the current step (that is, the view) from among the prior images registered in the database in association with a patient ID (identification information) of the current subject. However, this is merely an example, and the step identification information may be a combination of identification information of the protocol including the step and information (for example, an order of the step in the protocol) that uniquely identifies the step in the protocol. The information processing system 20 may display the image searched for in this way as the reference image 120.

In addition, for example, the information processing system 20 searches for the prior image that is highly related to the live image 110 from among the prior images for the current subject in the database. In this search, for example, a level of the relevance of the prior image to the live image 110 is calculated based on at least one of the level of the relevance of the attribute information between the ultrasound images or the image similarity between the ultrasound images. The image similarity is the similarity as an image between the images. The information processing system 20 may search for the image that is most highly related to the live image 110 and display the searched image as the reference image 120. In addition, as will be described in detail later, the prior image selected by the user from among a plurality of prior images that are highly related to the live image 110 and displayed in a prior image region 130 may be displayed as the reference image 120.

The user can adjust a position and an orientation of the probe 10 while referring to the displayed reference image 120. Further, the content of the displayed live image 110 can be evaluated while referring to the reference image 120.

In the example of FIG. 2, a size of the displayed reference image 120 is smaller than a size of the live image 110, but this is merely an example. The information processing system 20 may further have a display mode in which the reference image 120 is displayed in the same size as the live image 110.

The database in which the prior images are stored may be dedicated to the ultrasound diagnostic system 1 or may be a shared database accessible from the ultrasound diagnostic system 1. In this case, the shared database may be, for example, a patient database that records prior diagnostic information for each patient.

The prior image region 130 is provided below the live image 110. Thumbnails (that is, reduced images) 132 of the plurality of prior images that are highly related to the live image 110 are displayed in a list in the prior image region 130. One or more pieces of attribute information of the prior image, which are selected in advance, such as a date on which the prior image related to the thumbnail 132 is acquired may be displayed near the thumbnail 132 (for example, above or below the thumbnail). The information processing system 20 can receive selection from the user of an image to be displayed as the reference image 120 from among the thumbnails 132 of the prior images displayed in a list. In the present embodiment, processing of efficiently searching for the prior image that is highly related to the live image 110 is proposed. This processing will be described in detail later.

The thumbnail 132 has a smaller size than the reference image 120.

A current-examination image region 140 is provided on the display screen 100. Thumbnails 142 of current-examination images are displayed in a list in the current-examination image region 140. The current-examination image is an ultrasound image acquired during the current examination. For example, the ultrasound images acquired using the probe 10 from the start of the examination of the current target patient to the present and stored are the current-examination images. In addition, in a case of the examination using the protocol assistant function, the ultrasound images stored in each examination step up to the present in the protocol that is currently being executed are the current-examination images. One or more pieces of attribute information of the current-examination image, which are selected in advance, such as a name of the examination step in which the current-examination image related to the thumbnail 142 is acquired may be displayed near the thumbnail 142. The information processing system 20 can receive selection from the user of an image to be displayed as the reference image 120 from among the thumbnails 142 of the current-examination images displayed in a list.

In addition, as the reference image 120, a standard image may be displayed in addition to the prior image and the current-examination image described above. The standard image is a standard image that is not dependent on an individual and serves as a reference for the current examination, such as an ultrasound image or a schematic view of another person. The standard image is not an image acquired from the subject himself/herself. The user can select an image to be displayed as the reference image 120 from among the prior images displayed in a list in the prior image region 130, the current-examination images displayed in a list in the current-examination image region 140, a list of standard images, and the like.

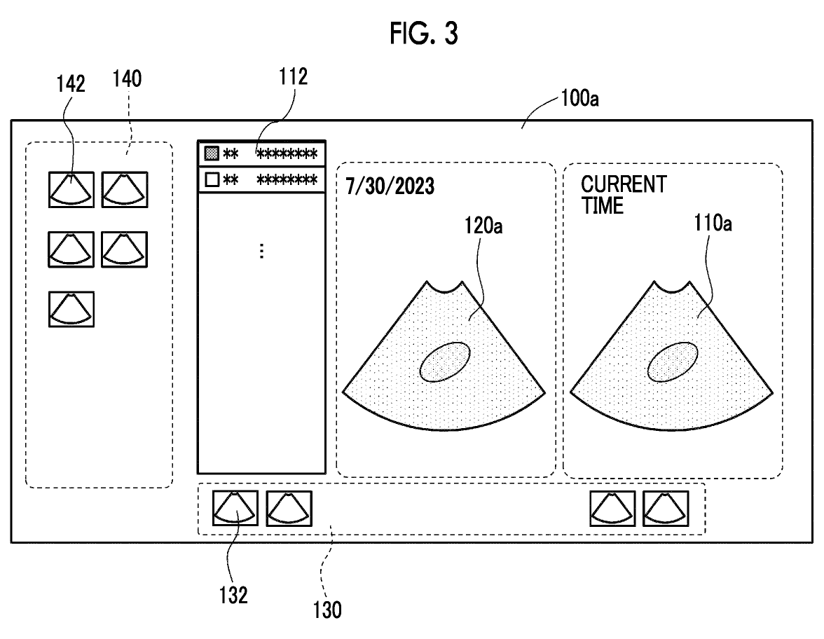

FIG. 3 schematically illustrates a display screen 100a having a different layout from FIG. 2. In the display screen 100a, a live image 110a and a reference image 120a are displayed side by side in the same size. This layout is referred to as a dual layout. On the other hand, the layout of the live image 110 and the reference image 120 illustrated in FIG. 2 is referred to as a standard layout. In the illustrated example, a size of the live image 110a in the dual layout is smaller than a size of the live image 110 in the standard layout, and a size of the reference image 120a in the dual layout is larger than a size of the reference image 120 in the standard layout.

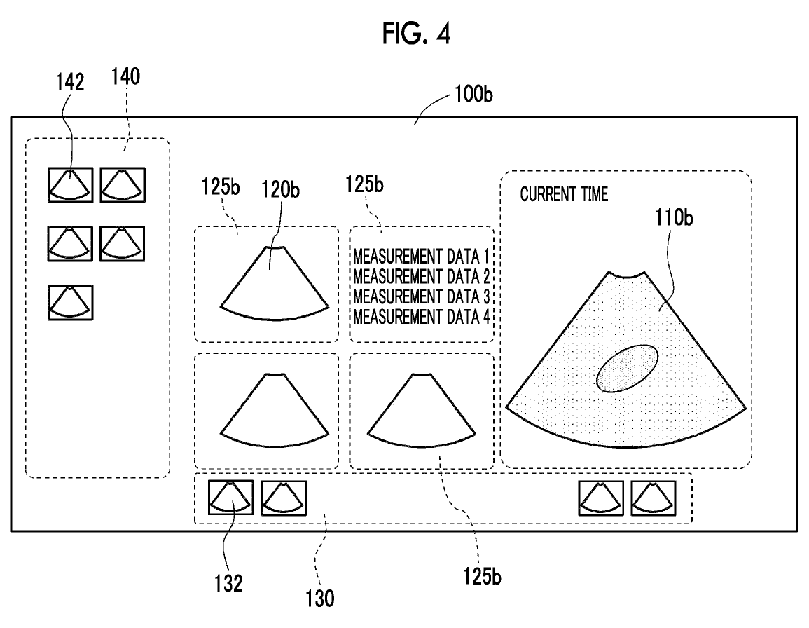

FIG. 4 schematically illustrates a display screen 100b having a different layout from the above. Four reference regions 125b arranged in a 2 × 2 array are provided on the display screen 100b. This layout is referred to as a multi-reference layout. A size of each reference region 125b is larger than a size of the thumbnail 132 or 142 and smaller than a size of a live image 110b. The 2 × 2 array described above is merely an example. The multi-reference layout generally refers to a layout in which there are a plurality of reference regions 125b.

A reference image 120b or other information can be displayed in each reference region 125b of the multi-reference layout. Examples of the "other information" displayed in the reference region 125b include measurement data. For example, in a case in which a certain prior image in a certain reference region 125b is displayed as the reference image 120b, one or more pieces of measurement data (for example, the flow velocity) corresponding to the prior image are displayed in the adjacent reference region 125b. In addition, another example of the "other information" is a graph (for example, a graph indicating a time-series change in measurement values obtained in a plurality of prior measurements) generated from the measurement data.

An information item (for example, the prior image, the measurement data, or the like) to be displayed in each reference region 125b of the multi-reference layout can be set in advance. In addition, the information processing system 20 may switch the display content of each reference region 125b displayed in accordance with the setting to another item based on an instruction from the user or the like.

The information processing system 20 receives an instruction from the user as to which of the display layouts illustrated in FIGS. 2 to 4 to use. Further, the information processing system 20 receives an instruction to change the display layout during the display.

Although not illustrated, an area for displaying the result of the measurement or a pop-up window may be provided on the display screen 100. The display of the result of the measurement may be the current result. Information (for example, the graph indicating a time-series change in measurement values) indicating a transition of the measurement result in each measurement from the prior measurement to the current measurement may be displayed. The information on the transition may indicate a change in the measurement result acquired in the same step of the same protocol for the same patient.

### Association Information

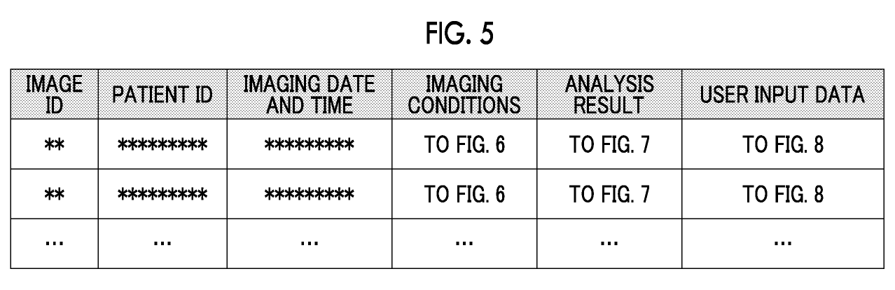

The association information associated with the ultrasound image will be described with reference to FIGS. 5 to 8. The association information is attribute information of the ultrasound image, and is registered in, for example, the database described above.

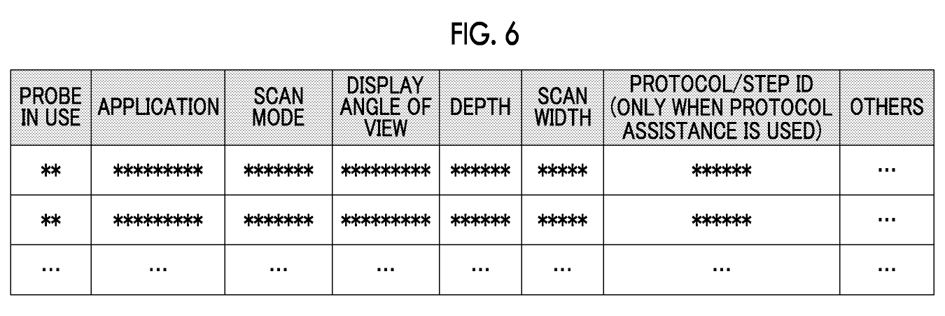

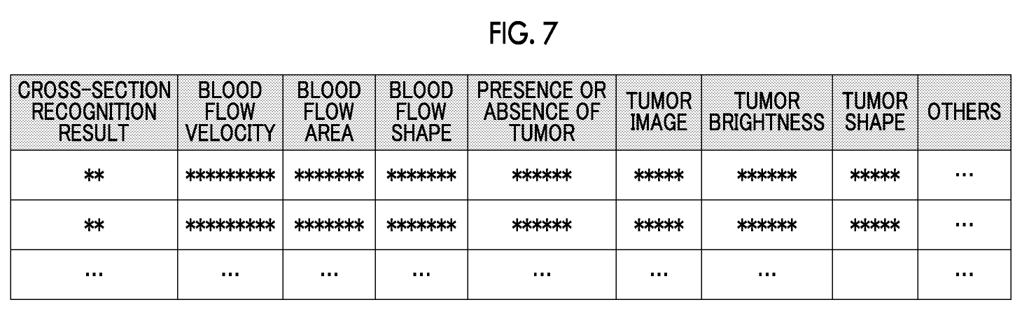

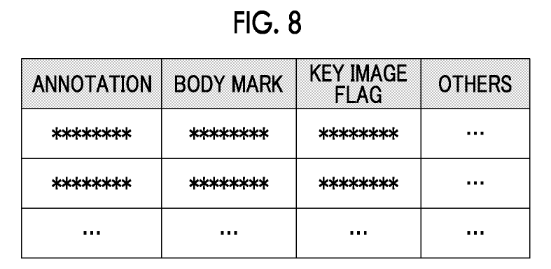

FIG. 5 illustrates an item group included in the association information. The association information illustrated in FIG. 5 includes items such as an image ID, a patient ID, an imaging date and time, imaging conditions, an analysis result, and user input data. The image ID is unique identification information assigned to the ultrasound image. The patient ID is unique identification information of the patient corresponding to the ultrasound image. The imaging date and time is a date and time when the ultrasound image is captured. The imaging conditions are various conditions (for example, a setting of an apparatus) in a case in which the ultrasound image is captured, and includes several items (will be described later with reference to FIG. 6). The analysis result is data of the results of various types of analyses or measurement on the ultrasound image, and includes several items (will be described later with reference to FIG. 7). The user input data is data input by the user in association with the ultrasound image, and includes several items (will be described later with reference to FIG. 8). The information such as the image ID, the patient ID, and the imaging date and time is acquired from a control mechanism of the ultrasound diagnostic system 1 and is registered in the database.

FIG. 6 illustrates an item group belonging to the imaging conditions in the association information. As illustrated in FIG. 6, the items belonging to the imaging conditions include a probe in use, an application, a scan mode, a display angle of view, a depth, a scan width, a protocol/step ID, and the like.

The "probe in use" is information indicating a type of the probe 10 used for acquiring the ultrasound image. Examples of the type of the probe 10 include a linear type, a sector type, and a convex type. In addition, for example, there are a plurality of probe models having different performance specifications even in the same linear type, and thus the "probe in use" may be information that can identify such a model level in a subdivided manner.

The "application" is an identification name of an application program used for generating, processing, or analyzing the ultrasound image. The "display angle of view" is, for example, a display angle of view of color Doppler display in the ultrasound image. The "depth" is a display width in a depth direction of the ultrasound image. The "scan width" is a width in a scan direction of the ultrasound image. The "protocol/step ID" is identification information indicating a protocol executed in a case of acquiring the ultrasound image and an examination step in the protocol. That is, in the examination using the protocol assistant function, identification information indicating a combination of the protocol and the examination step uniquely is recorded in the "protocol/step ID" for the ultrasound image acquired in the examination step in the protocol. In a case of the image acquired in the examination using no protocol assistant function, the "protocol/step ID" is left blank.

In addition to the examples illustrated in the drawing, information on a flow velocity setting in a case of a continuous wave mode or a pulsed-wave Doppler mode is also an example of the item of the imaging conditions.

In addition, the presence or absence of the measurement (for example, measurement of the blood flow velocity or diameter of the blood vessel) on the ultrasound image can also be the item of the imaging conditions.

The value of each item of the imaging conditions is acquired from the control mechanism of the ultrasound diagnostic system 1 and is registered in the database.

FIG. 7 illustrates an item group belonging to the analysis result in the association information. As illustrated in FIG. 7, the items belonging to the analysis result include a cross-section recognition result, a blood flow velocity, a blood flow area, a blood flow shape, the presence or absence of a tumor, a tumor image, a tumor brightness, and a tumor shape.

The "cross-section recognition result" is a recognition result of the cross section represented by the ultrasound image. This recognition result is obtained by the analysis processing of the cross-section recognition described above. The "blood flow velocity" is a flow velocity of the blood flow represented in the ultrasound image. This flow velocity is obtained by the analysis processing of the flow velocity detection described above. The "blood flow area" and the "blood flow shape" are information indicating an area and a shape of a blood flow region in the ultrasound image. These pieces of information can be obtained by known analysis processing. The "presence or absence of tumor" is information indicating whether or not a region corresponding to the tumor is included in the ultrasound image, and is obtained by the analysis processing of the tumor detection described above. The "tumor image" is an image of the region corresponding to the tumor in the ultrasound image, and is obtained by the analysis processing of the tumor detection described above. The "tumor brightness" and the "tumor shape" are information indicating a brightness and a shape of the region corresponding to the tumor, and these pieces of information can be obtained by known analysis processing.

Although not illustrated, in a case in which measurement other than the measurement of the blood flow velocity and the blood flow area (for example, measurement of a size of an object in the ultrasound image such as the blood vessel) is performed on the ultrasound image, the value of the measurement result is the item of the analysis result.

FIG. 8 illustrates an item group belonging to the user input data in the association information. As illustrated in the drawing, the items belonging to the user input data include an annotation, a body mark, a key image flag, and the like.

The "annotation" is an annotation input by the user for the ultrasound image. The annotation is an example of memo information input by the user. The "body mark" is a mark representing a location on a body of the subject where the probe 10 is applied. The user selects and inputs the body mark representing the position where the probe 10 is applied in the current examination from a list of the body marks provided by the ultrasound diagnostic system 1. The "key image flag" is binary data indicating whether or not the ultrasound image is a key image. The key image is an ultrasound image determined to be important by the user. In a case in which the user determines that the displayed ultrasound image is an image that is important for the diagnosis in the future, the user performs an operation of designating the ultrasound image as the key image. In response to the operation, the information indicating that the ultrasound image is the key image is registered in the database.

The item group that can be included in the association information of the ultrasound image has been described with reference to FIGS. 5 to 8. However, the item groups illustrated in FIGS. 5 to 8 are merely examples. The association information does not need to include all of the item groups illustrated in the drawings, and may include items not illustrated in the drawings. In addition, each ultrasound image does not need to have a value for all of the items of the association information, and may have a value of an item that can be obtained for the image among the items.

In addition, FIGS. 5 to 8 merely exemplify the item groups included in the association information, and do not define a data structure of the association information. For example, the grouping of the item groups of the imaging conditions, the analysis result, and the user input data illustrated in FIGS. 5 to 8 is merely for convenience of description. The database that manages the association information does not need to conform to such a grouping. In addition, the association information may be managed in a single database or may be managed in a distributed manner across a plurality of databases.

### Search for Related Prior Images

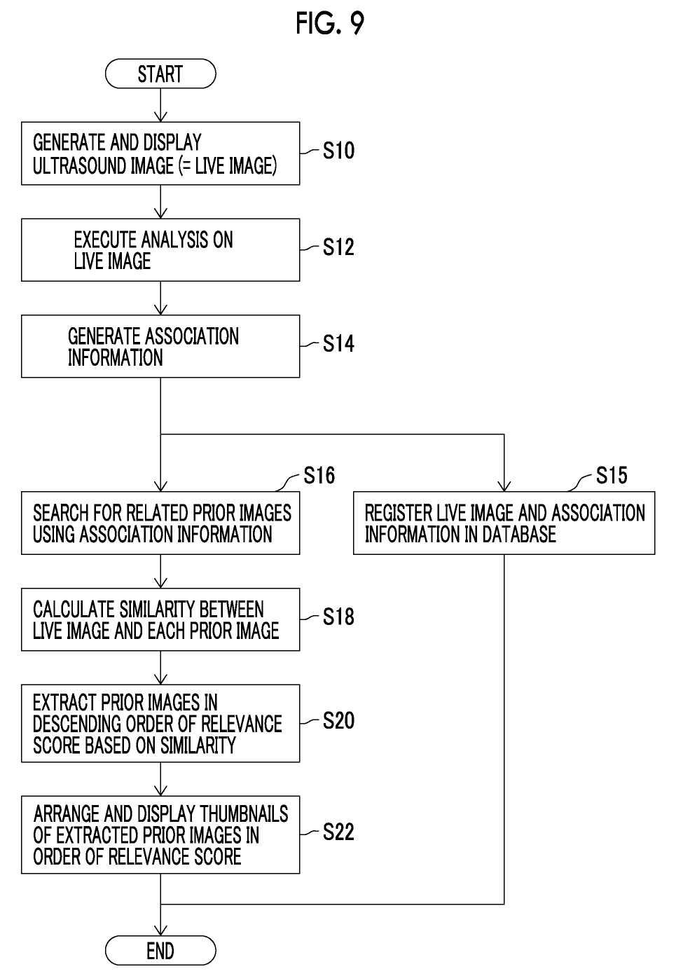

Next, a procedure of processing of searching for the prior image that is highly related to the live image 110 will be described with reference to FIG. 9.

In the procedure of FIG. 9, the information processing system 20 controls the transmit/receive unit 12 to cause the probe 10 to transmit and receive ultrasound, and causes the image forming unit 14 to generate the ultrasound image based on the reception signals obtained by the transmission and reception (S10). The ultrasound image generated in step S10 is displayed on the display screen 100 as the live image 110.

Next, the information processing system 20 executes the analysis processing described above on the live image 110 (S12). Further, the information processing system 20 acquires the value of each item of the imaging conditions of the live image 110. In a case in which the user inputs the annotation, the body mark, or the like to the display screen 100, the information processing system 20 acquires the input. Next, the information processing system 20 generates the association information for the live image 110 (S14). The association information includes a result of the analysis processing described above, the imaging conditions, and the data input by the user.

In a case in which the user inputs an instruction to store the live image 110, the information processing system 20 registers the live image 110 and the association information generated in step S14 in the database (S15). In this case, the association information registered in the database also includes the analysis result obtained by the analysis processing described above. Further, in this case, a unique image ID is assigned to the stored live image 110, and the image ID, the date and time at the corresponding point in time, and the patient ID of the current subject are registered in the database as a portion of the association information.

Here, a case has been described in which the user issues the instruction to store the live image 110, but in a case in which the information processing system 20 determines to store the live image 110 in accordance with a predetermined condition, the information processing system 20 also executes the processing of step S15 in the same manner. A timing when the information processing system 20 determines to store the live image can also be set in advance by the user. For example, a timing when the user issues an instruction to freeze (that is, pause) the live image 110 displayed in real time as the moving image may be determined as the timing to store the live image 110.

In addition, the registration of the association information in the database may be performed at any timing (for example, a timing when the user issues the instruction) after the examination ends.

In addition, the information processing system 20 searches the database for the prior image that is highly related to the live image 110 by using the association information obtained in S14 (S16). That is, the information processing system 20 searches for the prior image having the association information similar to the association information obtained in step S14 from among the prior images registered in the database in association with the patient ID of the current subject.

For example, in this search, the information processing system 20 searches for the prior image having high similarity to the value of the item in the association information of the live image 110 for the item for which the similarity can be defined. For example, the prior image having similarity equal to or higher than a threshold value or a predetermined number of prior images in descending order of the similarity is searched for. In addition, the information processing system 20 searches for the prior image having a value that matches the value of the item in the association information of the live image 110 for the item for which the similarity cannot be defined.

The association information generally includes both the item for which the similarity can be defined and the item for which the similarity cannot be defined. In this case, the information processing system 20 performs the search by, for example, the following procedure. That is, the information processing system 20 first searches for the prior image of which a value of the item for which the similarity cannot be defined matches the value of the corresponding item of the live image 110 from among the prior images in the database. In a case in which there are a plurality of items for which the similarity cannot be defined, the prior image of which the values of all of the plurality of items match the values of the corresponding items of the live image 110 is searched for. Then, the information processing system 20 searches for the prior image having high similarity to the value of the item of the live image 110 for the item for which the similarity can be defined from among the prior images included in the result of the search. In a case in which there are a plurality of items for which the similarity can be defined, the prior image having high overall score (that is, strong similarity) obtained by combining the similarities for each of the plurality of items is searched for.

In the above, in a case in which there are a plurality of items for which the similarity cannot be defined, the prior image of which the values of all of the plurality of items match the values of the corresponding items of the live image 110 is searched for, but an example in which the condition is relaxed can also be considered. For example, a condition may be set in which a predetermined percentage or more of the items need to match instead of requiring all of the items to match. Alternatively, in the item for which the similarity cannot be defined, an item for which the value match is required and an item for which the value match is not required may be defined in advance. In this case, the information processing system 20 searches for the prior image having a value that matches the value of the corresponding item of the live image 110 for all of the items for which the value match is required. The item for which the value match is not required may be excluded from the search condition. The information processing system 20 searches for the prior image satisfying the relaxed condition, and searches for the prior image having high similarity to the value of the item of the live image 110 for the item for which the similarity can be defined from among the prior images included in the result of the search.

The item for which the similarity can be defined is, for example, quantitative data or data that can be mapped to a metric space. For example, linguistic data such as a word, a phrase, a sentence, and a paragraph can be mapped to a vector space in which a distance can be defined, by being vectorized using a large-scale language model or the like. For example, the quantitative data such as the display angle of view, the depth, the scan width, the blood flow velocity, and the tumor brightness corresponds to the item for which the similarity can be defined. In addition, the linguistic data such as the annotation is also the item for which the similarity can be defined. In addition, the similarity as an image can be defined for the blood flow shape, the tumor shape, the tumor image, and the like. In addition, the cross-section recognition result is represented by a cross section name, but it is possible to predefine a degree of similarity between the cross-section names, for example, whether the cross-section names are cross sections of the same visceral organ, or the distance between the cross sections. Therefore, the cross-section recognition result can be regarded as the item for which the similarity can be defined.

Meanwhile, the item for which the similarity cannot be defined is a representative example of nominal scale data in statistics. The nominal scale is a name or the like having a meaning only for distinguishing the data from the other data. For example, the probe in use, the scan mode, the presence or absence of the measurement, and the like correspond to the item for which the similarity cannot be defined.

As described above, in step S16, the information processing system 20 narrows down the prior images close to the live image 110 in terms of the association information.

Next, the information processing system 20 calculates the image similarity between each prior image searched for in step S16 and the live image 110 (S18). Various known methods can be used for the calculation of the image similarity. Among these methods, there is, for example, a method of obtaining a feature value such as a vector representing a feature of the image and obtaining the similarity between the feature values between the live image 110 and the prior image.

In addition, in step S18, the information processing system 20 calculates a relevance score for each of the prior images based on the image similarity calculated above. The relevance score is a value representing the level of the relevance of the prior image to the live image 110. For example, the relevance score becomes a larger value as the prior image is more highly related to the live image 110. In one example, the relevance score may be the image similarity calculated in step S18 itself. In another example, the relevance score may be calculated based on the image similarity calculated in step S18 and the overall score of the similarities of each item of the association information described above. In the calculation of the overall score, points may be added or deducted in accordance with the similarity of the value of the item between the live image 110 and the prior image for one or more items of the association information. For example, the amount of added points is increased as the similarity becomes higher, and points are deducted as the similarity becomes lower to a certain extent or more. As a specific example, the amount of added points may be increased as the difference in flow velocity setting value between the live image 110 and the prior image becomes smaller.

The information processing system 20 extracts several prior images in descending order of the relevance score calculated in step S18 from among the prior images searched for in step S16 as images that are highly related to the live image 110 (S20). In this step, for example, the prior image having a relevance score equal to or higher than a predetermined threshold value is extracted. In another example, in this step, a predetermined number of prior images are extracted in descending order of the relevance score.

Then, the information processing system 20 displays the thumbnails 132 of the prior images extracted in step S20 in the prior image region 130 of the display screen 100 side by side in descending order of the relevance score (S22).

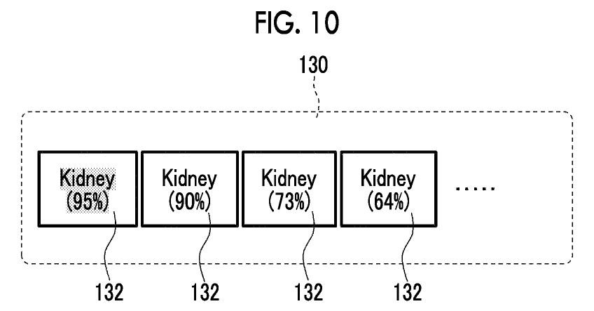

FIG. 10 schematically illustrates an example of the prior image region 130 displayed in step S22. In this example, the thumbnails 132 of the prior images extracted in step S20 are arranged in the prior image region 130 in descending order of the relevance score from left to right. In the illustrated prior image region 130, for the live image 110 representing a specific cross section of a liver of a certain subject, the thumbnails 132 of the prior images of the liver of the same subject that are highly related to the live image 110 are arranged. In the illustrated example, the relevance scores of the prior images represented by the thumbnails 132 with respect to the live image 110 are 95%, 90%, 73%, 64%, ... in order from the left. Although not illustrated, the value of one or more specific items of the association information, such as the imaging date of the prior image represented by the thumbnail 132, may be displayed near each thumbnail 132 (for example, below the thumbnail 132).

In a case in which the thumbnails 132 of the prior images are displayed in the prior image region 130 in accordance with the procedure of FIG. 9, the information processing system 20 may display the prior image having a high relevance score among the thumbnails 132 as the reference image 120 on the display screen 100.

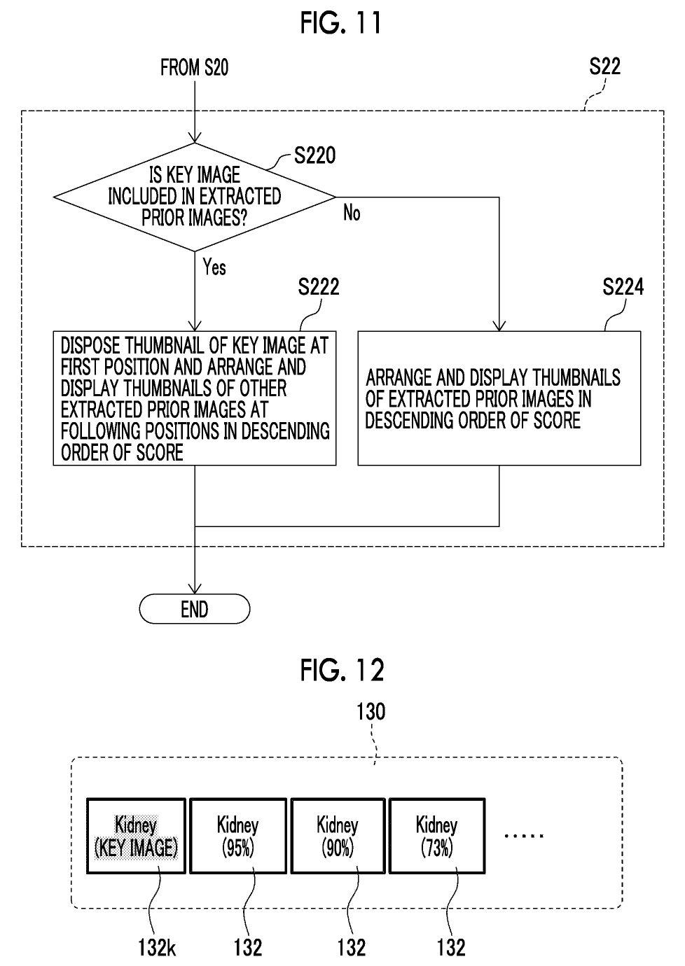

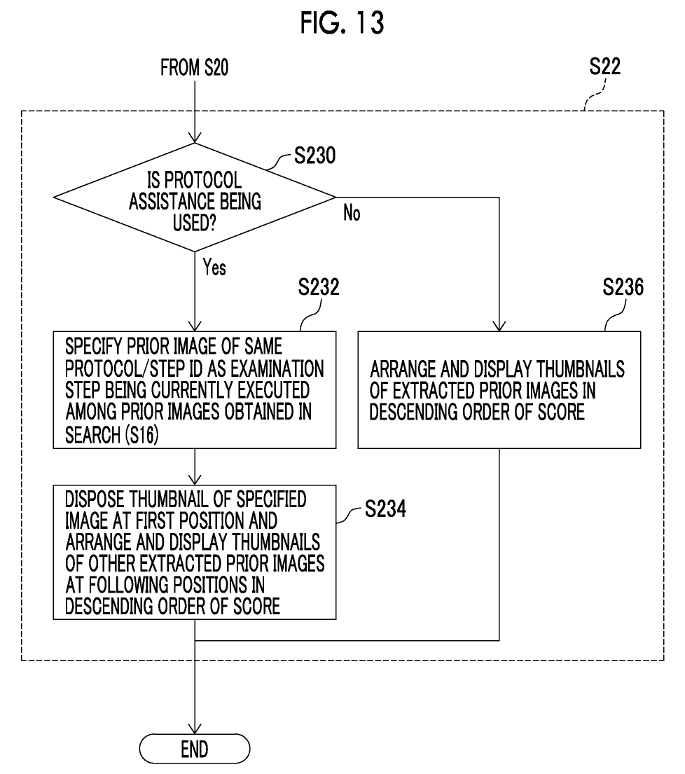

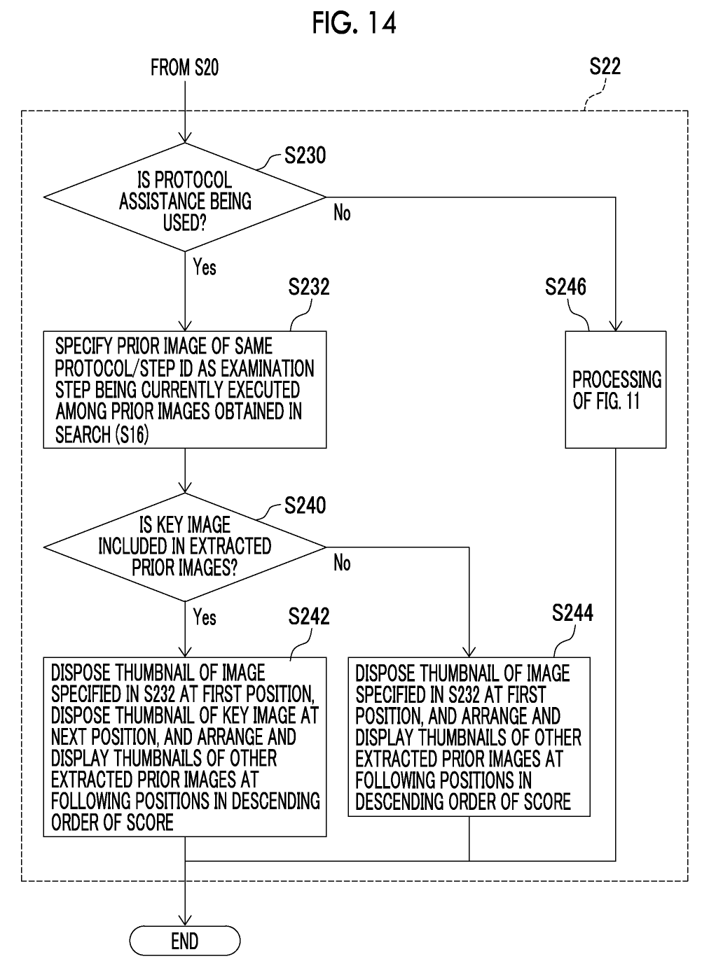

In a case of taking the key image into account, step S22 may be a procedure illustrated in FIG. 11, for example. That is, the information processing system 20 determines whether or not the key image is included in the prior images extracted in step S20 (S220). This determination may be performed by checking a key information flag (see FIG. 8) of the association information of the prior images. In a case in which the determination result of step S220 is Yes, the information processing system 20 disposes the key image among the prior images at the first position of the prior image region 130, and arranges and displays the other prior images at the following positions in descending order of the relevance score (S222).

FIG. 12 illustrates an example of the prior image region 130 displayed in step S222. In the example of FIG. 12, a thumbnail 132k of the key image is disposed at the left end of the prior image region 130, and the thumbnails 132 of the other prior images are arranged to the right of the thumbnail 132k in descending order of the relevance score.

In this way, in step S222, the thumbnail 132k of the key image is disposed at the first position of the prior image region 130 regardless of the value of the relevance score.

In a case in which the determination result of step S220 is No, the information processing system 20 performs the same processing as step S22 of FIG. 9. That is, the information processing system 20 displays the thumbnails 132 of the prior images extracted in step S20 in the prior image region 130 side by side in descending order of the relevance score (S224).

As described above, in the examples of FIGS. 11 and 12, since the thumbnail 132k of the key image is disposed at the first position (in FIG. 12, the leftmost position) of the prior image region 130, it is easy to find the thumbnail 132k.

In addition, in this example, the prior image corresponding to the thumbnail disposed at the first position of the prior image region 130 in step S222 may be automatically displayed as the reference image 120.

### Example of Control Depending on Whether or Not Protocol Assistant Function Is Used

A partially modified procedure of the procedure of FIG. 9 that can be applied in a case of taking the protocol assistant function into account will be described with reference to FIG. 13. FIG. 13 illustrates a portion that can be substituted for step S22 of FIG. 9.

The information processing system 20 determines, for example, after step S20, whether or not the protocol assistant function is used in the current examination (S230). In a case in which the determination result of step S230 is Yes, the information processing system 20 specifies the prior image having the same protocol/step ID as the protocol/step ID of the examination step that is currently being executed from among the prior images obtained by the search in step S16 (S232). Then, the information processing system 20 disposes the thumbnail of the prior image specified in step S232 at the first position of the prior image region 130, and arranges and displays the thumbnails 132 of the other prior images extracted in step S20 at the following positions in descending order of the relevance score (S234). In a case in which a plurality of prior images are specified in step S232, the information processing system 20 arranges the plurality of specified prior images in the prior image region 130 in descending order of the relevance score from the first position, and arranges the other prior images at the following positions.

Although not illustrated to avoid complication, it is also possible that the prior image having the same protocol/step ID as the protocol/step ID of the examination step that is currently being executed is not specified in step S232. In this case, in step S234, the information processing system 20 simply displays the thumbnails 132 of the prior images extracted in step S20 in the prior image region 130 side by side in descending order of the relevance score.

In a case in which the protocol assistant function is not used (that is, the determination result of step S230 is No), the information processing system 20 displays the thumbnails 132 of the prior images extracted in step S20 in the prior image region 130 side by side in descending order of the relevance score (S236).

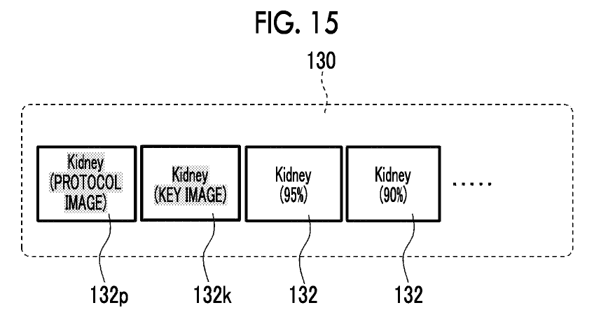

In addition, in a case in which both the key image and the protocol assistant function are taken into account, step S22 of FIG. 9 may be changed as illustrated in FIG. 14. In FIG. 14, the same reference numerals are given to the steps that are the same as the steps included in the procedure of FIG. 13. In addition, FIG. 15 illustrates an example of the prior image region 130 displayed in step S242.

In this example, in a case in which the protocol assistant is currently being used, the information processing system 20 specifies the prior image having the same protocol/step ID as the protocol/step ID of the examination step that is currently being executed from the search result of step S16 (S232). Further, the information processing system 20 determines whether or not the key image is included in the prior images extracted in step S20 (S240). In a case in which the determination result is Yes, the information processing system 20 disposes a thumbnail 132p of the prior image specified in step S232 at the first position from the left end of the prior image region 130, and disposes the thumbnail 132k of the key image found in step S240 adjacent to the right of the thumbnail 132p (see FIG. 15). Then, the thumbnails 132 of the other prior images extracted in step S20 are arranged and displayed in descending order of the relevance score (S242).

In a case in which the determination result of step S240 is No, the information processing system 20 disposes the thumbnail 132p of the prior image specified in step S232 at the first position in order from the left end of the prior image region 130. Then, the thumbnails 132 of the other prior images extracted in step S20 are arranged and displayed in descending order of the relevance score (S244).

In a case in which it is determined in step S230 that the protocol assistant function is not used, the information processing system 20 executes the processing procedure of FIG. 11 (S246).

In addition, while a certain examination step of a certain protocol is being executed using the protocol assistant function, an additional examination may be performed. For example, the user who has observed the ultrasound image obtained in the examination step that is currently being executed may interrupt the execution of the protocol and instruct an additional examination (for example, ultrasound imaging in another mode) that is not included in the examination step in order to examine it in more detail. In such a case, in a case in which the ultrasound image of the additional examination associated with the same protocol/step ID as the interrupted examination step is included in the prior image obtained in the previous examination of the same patient, the information processing system 20 may increase the score of the image. It is considered that the additional examination previously performed in the same examination step is likely to examine the same part or lesion as the current additional examination, so that the score of the image of the prior additional examination can be increased to increase the display rank of the thumbnail of the image in the prior image region 130.

### Example of Control Taking Tumor into Account

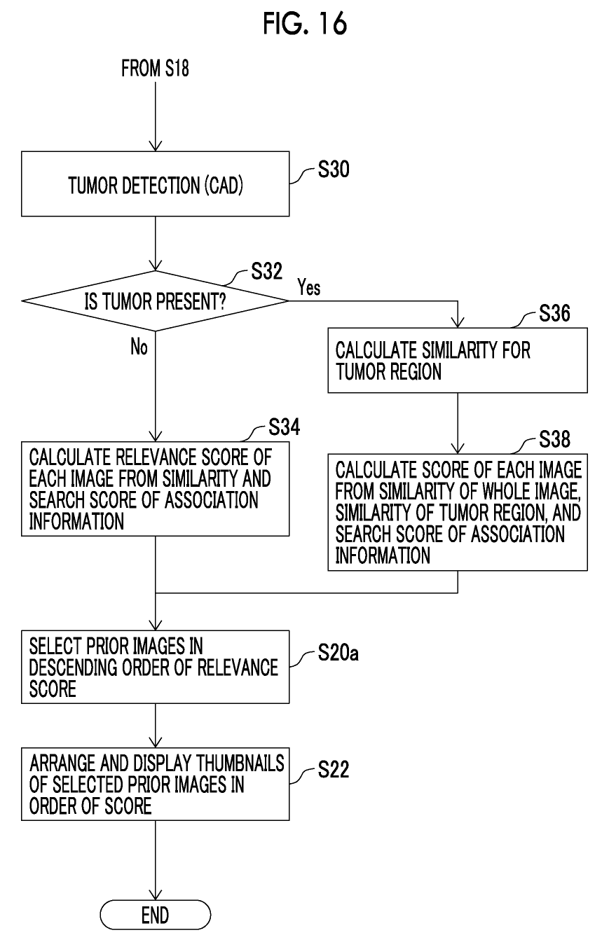

Next, a modification example of the processing procedure of FIG. 9 will be described with reference to FIG. 16. In this modification example, the search result is ranked by also taking into account the image similarity between the tumor regions of the live image 110 and the prior image. FIG. 16 illustrates processing of a portion after step S18 in the procedure of FIG. 9.

In this modification example, the information processing system 20 executes the tumor detection on the live image 110 using a system such as CAD (S30). In a case in which the region corresponding to the tumor is not detected in the live image 110 in the tumor detection (determination result of S32 is No), the information processing system 20 calculates the relevance score of each prior image based on the image similarity calculated in step S18 and the search score in which the similarities of each item of the association information are integrated (S34). Next, the information processing system 20 extracts several prior images in descending order of the relevance score from among the prior images searched for in step S16 as the images that are highly related to the live image 110 (S20a). Then, the information processing system 20 displays the thumbnails 132 of the prior images extracted in step S20a in the prior image region 130 of the display screen 100 side by side in descending order of the relevance score (S22).

In a case in which the determination result of step S32 is Yes, the information processing system 20 calculates the image similarity between the prior image and the tumor region in the live image 110 detected in S30 for each prior image searched for in step S16 (S36). In this calculation, for example, for each of the searched prior images, a region that is most similar to the tumor region in the prior image is searched for, and the image similarity between the region found by the search and the tumor region in the live image 110 is calculated. This calculation may be performed by, for example, a method such as template matching in which the image of the tumor region in the live image 110 is used as a template.

Next, the information processing system 20 calculates the relevance score of each prior image based on the image similarity of the whole image calculated in step S18, the image similarity for the tumor region calculated in step S36, and the search score in which the similarities of each item of the association information are integrated (S38). Then, the information processing system 20 extracts several prior images in descending order of the relevance score (S20a), and displays the thumbnails 132 of the extracted prior images in the prior image region 130 side by side in descending order of the relevance score (S22).

### Example Taking Moving Image into Account

The live image 110 displayed on the display screen 100 is generally a moving image. The user can set the live image 110 on the display screen 100 to a still image by pausing the moving image or advancing frame by frame to select a specific frame. In one example, the information processing system 20 executes the procedure of FIG. 9 with the live image 110 as the still image determined by the user in this way as the target. In addition, the information processing system 20 may select a representative frame from the displayed moving image and execute the procedure of FIG. 9 with the selected frame as the live image 110.

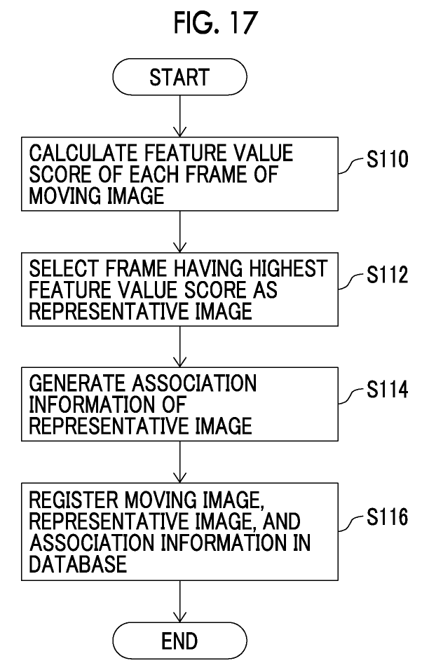

Further, the information processing system 20 may have a function of registering the moving image in the database. For example, in a case in which the user issues an instruction to register the moving image that is currently being displayed in the database, the information processing system 20 executes the procedure illustrated in FIG. 17.

In the procedure of FIG. 17, the information processing system 20 calculates a feature value score for the image of each frame of the moving image to be registered in the database (S110). The feature value score is an index value indicating a degree of contribution of the image of the frame to the diagnosis.

For example, an amount of blurring of the image of the frame may be obtained as one item of the feature value. In this case, the feature value score may be defined such that the feature value score of the frame becomes higher as the amount of blurring becomes smaller.

In addition, a recognition rate of the visceral organ from the image of the frame may be obtained as one item of the feature value. The recognition rate of the visceral organ can be obtained by, for example, a scene analysis. In this case, the feature value score may be defined such that the feature value score of the frame becomes higher as the recognition rate becomes higher. In addition, an amount of missing of a visceral organ region in the image of the frame may be obtained as one item of the feature value. In this case, the feature value score may be defined such that the feature value score of the frame becomes higher as the amount of missing becomes larger. The missing of the visceral organ region in the image is caused by, for example, a shadow of a bone.

In addition, whether or not the frame includes the region having the characteristics of the tumor may be obtained as one item of the feature value. In this case, the feature value score may be defined such that the feature value score becomes higher in a case in which the region having the characteristics of the tumor is included than in a case in which the region having the characteristics of the tumor is not included. The region having the characteristics of the tumor is obtained by, for example, an AI-based tumor detection function such as the above-described CAD. In addition, a size of the region having the characteristics of the tumor in the frame may be obtained as one item of the feature value. In this case, the feature value score may be defined such that the feature value score becomes higher as the region having the characteristics of the tumor becomes larger. In addition, a confidence level for the region having the characteristics of the tumor may be obtained as one item of the feature value. As the confidence level, a value of the confidence level output in a case in which the tumor detection function detects the region having the characteristics of the tumor may be used.

In addition, blood-flow sensitivity may be obtained as one item of the feature value. The feature value score may be defined such that the feature value score becomes higher as the blood-flow sensitivity becomes higher. In addition, an amount of blood-flow aliasing may be obtained as one item of the feature value. The feature value score may be defined such that the feature value score becomes higher as the amount of blood-flow aliasing becomes smaller. The blood-flow sensitivity and the amount of blood-flow aliasing can be determined, for example, by evaluating the color distribution of the blood-flow information shown in the color flow image and by performing texture analysis.

The examples of some feature value items that form the basis of the feature value score and the relationship between the values of these items and the feature value score have been described above. These are merely examples. It is not always necessary to use all of these items. In addition, one or more items other than these items may be reflected in the feature value score.

In a case in which the feature value score of each frame in the moving image is calculated, the information processing system 20 selects the frame having the highest feature value score as the representative image (S112). The representative image is a still image that is representative of the moving image. The information processing system 20 performs the analysis processing described above on the representative image to generate the association information including the result of the analysis (S114). Among the items of the association information, the items calculated in step S110 are used as the calculation result.

Then, the information processing system 20 registers the moving image to be registered, the representative image selected in step S112, and the association information generated in step S114 in the database in association with each other (S116).

In a case of taking into account a case in which the moving image is registered in the database in this way, in the processing of searching for the prior image in the procedure of FIG. 9, the representative image is the target of the processing instead of the moving image. That is, in step S18 of FIG. 9, the information processing system 20 calculates the image similarity between the live image 110 and the representative image for an entry (hereinafter, referred to as a moving image entry) in which a set of the moving image, the representative image, and the association information is registered in the database, and calculates the relevance score based on the image similarity. In addition, in a case in which the representative image is extracted in step S20 based on the relevance scores calculated in this way, the thumbnail of the representative image is displayed in the prior image region 130 in step S22. In addition, in a case in which the thumbnail of the representative image displayed in the prior image region 130 is selected by the user, the information processing system 20 displays the representative image or the moving image corresponding to the representative image as the reference image 120. The display of the representative image or the moving image in the reference image 120 can be switched in accordance with the instruction of the user.

In addition, a plurality of visceral organs may be displayed in one moving image. In such a case, the frame in which the feature of the visceral organ is well represented is not infrequently different for each visceral organ. Therefore, the representative image may be selected individually for each of the plurality of visceral organs. For example, for each visceral organ, the frame having the highest feature value score for the visceral organ may be selected as the representative image. Each visceral organ displayed in each frame of the moving image can be recognized by the analysis processing described above.

### Other Modification Examples

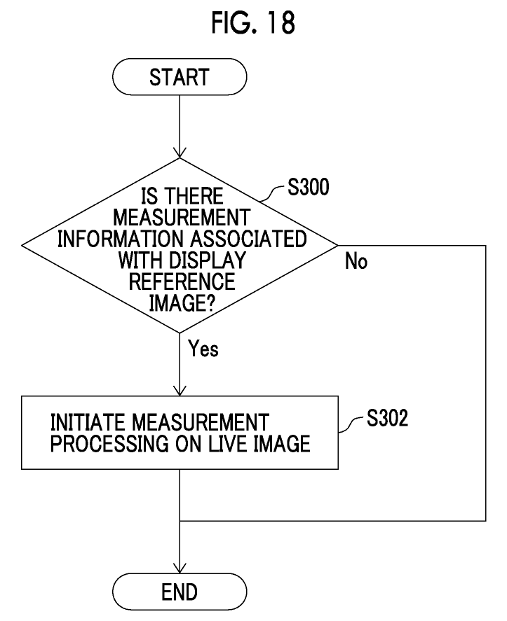

Another modification example will be described with reference to FIG. 18. The procedure illustrated in FIG. 18 is executed at a timing when the information processing system 20 newly displays the reference image 120 or switches the displayed reference image 120 to another image.

In the procedure of FIG. 18, the information processing system 20 determines whether or not the association information corresponding to the displayed reference image 120 includes measurement information (S300). The measurement information includes, for example, information indicating a measured item (that is, a type of measurement) and a value of the measurement result. In a case in which the determination result of step S300 is Yes, the information processing system 20 initiates processing of performing measurement corresponding to the measurement information for the current examination (S302). The processing of performing the measurement is, for example, the measurement of the item indicated by the measurement information itself. In another example, the processing of performing the measurement may include other types of processing (for example, input of information necessary for the measurement) required to execute the measurement of the item.

In step S302, the information processing system 20 automatically executes, for example, the measurement of the measurement item indicated by the measurement information. For example, in a case in which the live image 110 is the blood-flow velocity waveform in the PW mode and the measurement item included in the measurement information is the maximum value of the blood flow velocity, the information processing system 20 calculates the maximum value of the blood flow velocity indicated by the waveform of the live image 110.

In another example, in step S302, the information processing system 20 executes processing of requesting the user to input information necessary for executing the measurement item. In a case in which the necessary information is input, the information processing system 20 executes the measurement using the information and records the measurement result.

For example, in a case in which the association information of the reference image 120 includes a measurement result of a length of a tissue, the information processing system 20 displays reference points for specifying both ends of the part to be measured for the length on the live image 110. The user moves the reference points to appropriate positions. After the positions of the reference points are appropriately set, the information processing system 20 measures a distance between the two reference points and records the distance.

In a case in which the determination result of step S300 is No, the information processing system 20 skips step S302 and ends the processing.

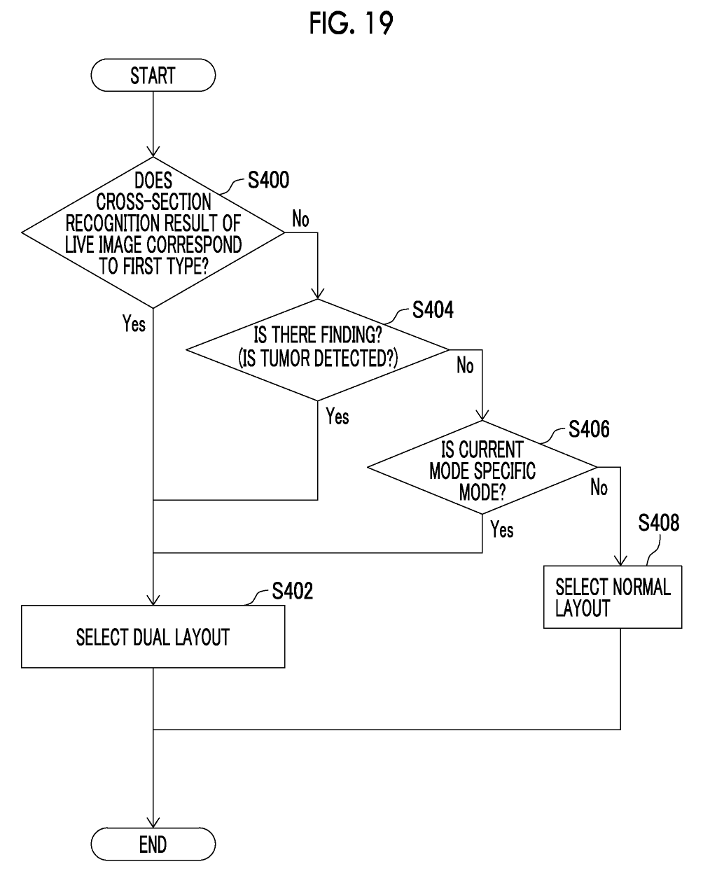

Still another modification example will be described with reference to FIG. 19. FIG. 19 illustrates an example of a procedure in which the information processing system 20 automatically selects the layout of the display screen 100.

As a premise of this procedure, the cross sections are classified into two types: a first type and the other type. In a case in which measurement is defined for the ultrasound image of the cross section, the cross section corresponds to the first type. In addition, in a case in which the comparison of the shape between the prior image of the same cross section is defined for the ultrasound image of the cross section, the cross section corresponds to the first type. Whether or not each cross section corresponds to the first type is registered in the information processing system 20 in advance.

The procedure of FIG. 19 is executed, for example, after the analysis processing of step S12 of the procedure of FIG. 9.

In this procedure, the information processing system 20 determines whether or not the cross-section recognition result (which is obtained by the analysis processing) for the live image 110 corresponds to the first type (S400). In a case in which the determination result is Yes, the information processing system 20 selects the dual layout (see FIG. 3) as the layout of the display screen 100 (S402).

On the other hand, in a case in which the determination result of step S400 is No, the information processing system 20 determines whether or not the finding is generated for the live image 110 by the analysis processing (S404). In a case in which the determination result of step S404 is Yes, the information processing system 20 selects the dual layout (S402).

In addition, in a case in which the determination result of step S404 is No, the information processing system 20 determines whether or not a current mode of the ultrasound diagnostic system 1 is a specific mode (S406). In a case in which the determination result is Yes, the information processing system 20 selects the dual layout (S402).

Here, the "specific mode" includes a continuous wave Doppler mode, a pulsed-wave Doppler mode, a power Doppler mode (for example, eFlow), a tissue Doppler imaging (TDI) mode, a detective Flow imaging (DFI) mode, and the like. For example, in the continuous wave Doppler or pulsed-wave Doppler mode, a waveform indicating a time-series change in the blood flow velocity is displayed, but the image of such a waveform can obtain useful information by comparing the image with the image of the prior waveform. In addition, it is important to diagnose how the shape or the texture represented by the image has changed from the prior time in the image obtained by the power Doppler, the TDI, or the DFI. In a normal layout (FIG. 2) in which the reference image 120 is smaller than the live image 110, it is difficult to compare the live image 110 with the reference image 120 that is the prior image. For this reason, in a case of corresponding to the specific mode, the dual layout in which the live image 110 and the reference image 120 are easily compared is adopted.

In a case in which the determination result of step S406 is No, the information processing system 20 selects the normal layout (see FIG. 2) as the layout of the display screen 100 (S408).

After step S402 or S408, the information processing system 20 generates the display screen 100 of the selected layout and displays the display screen 100.

As described above, in this modification example, in a case in which the live image 110 for the cross section for which the measurement or the comparison with the prior image is necessary is displayed, the live image 110 is displayed in the dual layout. In the dual layout, since the reference image 120 is displayed larger than in the normal layout, a position of a measurement cursor in the reference image 120 is easy to see. In addition, in the dual layout, since the live image 110 and the reference image 120 are displayed in the same size, it is easy for the user to compare the shapes of the two images.

In addition, in this modification example, in a case in which the tumor or the like is detected from the live image 110, since the reference image 120 and the live image 110 are displayed in the same size, it is easy to compare the reference image 120 and the live image 110. By this comparison, it is possible to recognize how the size or the property of the tumor or the like has changed from the state at the prior time represented by the reference image 120.

In this example, the display of the dual layout is performed in a case in which the finding is generated. A case in which the finding is generated by the information processing system 20 is a case in which a part suspected of having some abnormality is found in the live image 110. In such a case, the user can compare the two images to check the presence or absence of the abnormality and the state by displaying the reference image 120 in the same size as the live image 110 in the dual layout.

In the present embodiment, each processing is executed by any computer. Further, any computer may execute these types of processing by a processor as hardware, a program as software, or a combination thereof. In such a case, the processor is configured to execute various types of processing in the present embodiment in cooperation with the program, and may function as each unit or each means in the present embodiment. In addition, the execution order of the processing by the processor is not limited to the above-described order and may be changed as appropriate. Any computer may be a general-purpose computer, a dedicated computer, a workstation, or another system that can execute each processing.

The processor may be configured by one or more hardware components, and the type of hardware is not limited. For example, the processor may be implemented by hardware such as a programmable logic device, for example, a central processing unit (CPU), a micro processing unit (MPU), or a field programmable gate array (FPGA), a dedicated circuit for executing specific processing such as an application specific integrated circuit (ASIC), a graphics processing unit (GPU), or a neural processing unit (NPU). Moreover, the type of hardware may be a combination of different types of hardware. In a case in which the plurality of types of hardware are configured to execute one or a plurality of types of processing of a certain processor, the plurality of types of hardware may be present in devices physically separated from each other or may be present in the same device. Furthermore, in any of the embodiments, the order of each type of processing executed by the processor is not limited to the above-described order, and may be changed as appropriate. In addition, hardware is implemented in a form of an electric circuit (circuitry) in which circuit elements, such as semiconductor elements, are combined.

Further, the program may be software such as firmware or microcode. The program may be, for example, a group of program modules, and each function thereof may be implemented by a processor configured to execute each function. The program may be a program code or a plurality of code segments stored in one or more non-transitory computer-readable media (for example, a storage medium and other storages). The program may be stored in the plurality of non-transitory computer-readable media present in devices physically separated from each other. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, arguments, parameters, or contents in the memory.

What is claimed is:

1. An ultrasound diagnostic system comprising:
a processor configured to:
generate a first ultrasound image from reception signals of an ultrasound probe;
execute processing of displaying the first ultrasound image;
execute an analysis of the first ultrasound image to obtain an analysis result;
execute a search for prior ultrasound images having analysis results that are the same as or similar to the analysis result for the first ultrasound image;
calculate a score representing similarity to the first ultrasound image for each of the prior ultrasound images obtained as a result of the search; and
execute display control of displaying, together with the first ultrasound image, one or more images selected based on the scores from among the prior ultrasound images obtained as a result of the search.

2. The ultrasound diagnostic system according to claim 1,
wherein the analysis is processing of obtaining, as the analysis result, a cross-section recognition result indicating which of a plurality of predetermined cross sections the first ultrasound image corresponds to.

3. The ultrasound diagnostic system according to claim 1,
wherein the first ultrasound image is an image of a blood-flow velocity waveform obtained by a Doppler method, and
the analysis is processing of obtaining, as the analysis result, a maximum flow velocity of the blood-flow velocity waveform.

4. The ultrasound diagnostic system according to claim 1,
wherein the processor is configured to:
execute processing of detecting a tumor region from the first ultrasound image; and
calculate, in the calculation of the scores, the score between the prior ultrasound image and the first ultrasound image based on first similarity between a whole prior ultrasound image and a whole first ultrasound image and second similarity between a tumor region of the prior ultrasound image and the tumor region of the first ultrasound image.

5. The ultrasound diagnostic system according to claim 1,
wherein the processor is configured to:
in a case of storing the first ultrasound image, store the analysis result obtained by the analysis as one item of attribute information of the first ultrasound image.

6. The ultrasound diagnostic system according to claim 1,
wherein the processor is configured to:
in a case in which the first ultrasound image is a moving image, store the moving image, a still image that is representative of the moving image, and an analysis result obtained for the still image by the analysis.

7. The ultrasound diagnostic system according to claim 1,
wherein the processor is configured to:
in the search, acquire memo information input by a user for the displayed first ultrasound image, and search for prior ultrasound image data having an analysis result that is the same as or similar to the obtained analysis result and having memo information that is the same as or similar to the acquired memo information.

8. The ultrasound diagnostic system according to claim 1,
wherein the processor is configured to:
in the display control, execute processing of displaying thumbnails of a plurality of images selected based on the scores from among the prior ultrasound images obtained as a result of the search side by side in descending order of the score in a thumbnail display field provided near the first ultrasound image.

9. The ultrasound diagnostic system according to claim 8,
wherein the processor is configured to:
in the display control, in a case in which an image designated as a key image is included in the plurality of images selected based on the scores from among the prior ultrasound images obtained as a result of the search, display a thumbnail of the image designated as the key image at a first position of the thumbnail display field regardless of the score for the image.

10. The ultrasound diagnostic system according to claim 9,
wherein the processor is configured to:
in a case in which a protocol assistant function of supporting execution of a protocol consisting of one or more examination steps is used, in the display control, display a thumbnail of a prior ultrasound image obtained in the same examination step of the same protocol as an examination step of a protocol that is currently being executed, at the first position of the thumbnail display field, and display the thumbnail of the image designated as the key image at a next position.

11. The ultrasound diagnostic system according to claim 8,
wherein the processor is configured to:
in a case in which a protocol assistant function of supporting execution of a protocol consisting of one or more examination steps is used, in the display control, display a thumbnail of a prior ultrasound image obtained in the same examination step of the same protocol as an examination step of a protocol that is currently being executed, at a first position of the thumbnail display field.

12. The ultrasound diagnostic system according to claim 1,
wherein the processor is configured to:
in the display control, display the prior ultrasound image having a highest score side by side with the first ultrasound image, as a reference image for the first ultrasound image.

13. The ultrasound diagnostic system according to claim 12,
wherein the processor is configured to:
in a case in which information indicating a measurement performed on the prior ultrasound image displayed as the reference image is obtained, execute processing of performing the measurement on the displayed first ultrasound image.

14. The ultrasound diagnostic system according to claim 2,
wherein the processor is configured to:
select a layout for displaying the first ultrasound image and a reference image selected from among the prior ultrasound images, based on the cross-section recognition result.

15. The ultrasound diagnostic system according to claim 14,
wherein the processor is configured to:
in a case in which the cross-section recognition result corresponds to a predetermined first type of cross section, select a dual layout in which the first ultrasound image and the reference image are arranged in the same size.

16. The ultrasound diagnostic system according to claim 1,
wherein the processor is configured to:
in a case in which a finding is generated by the analysis, as a layout for displaying the first ultrasound image and a reference image selected from among the prior ultrasound images, select a dual layout in which the first ultrasound image and the reference image are arranged in the same size.

17. The ultrasound diagnostic system according to claim 1,
wherein the processor is configured to:
in a case in which a mode of the ultrasound diagnostic system when the first ultrasound image is generated is a specific mode, as a layout for displaying the first ultrasound image and a reference image selected from among the prior ultrasound images, select a dual layout in which the first ultrasound image and the reference image are arranged in the same size.

18. A method for operating an ultrasound diagnostic system comprising:
generating a first ultrasound image from reception signals of an ultrasound probe;
executing processing of displaying the first ultrasound image;
executing an analysis of the first ultrasound image to obtain an analysis result;
executing a search for prior ultrasound images having analysis results that are the same as or similar to the analysis result for the first ultrasound image;
calculating a score representing similarity to the first ultrasound image for each of the prior ultrasound images obtained as a result of the search; and
executing display control of displaying, together with the first ultrasound image, one or more images selected based on the scores from among the prior ultrasound images obtained as a result of the search.
